(19) United States
(12) Patent Application Publication
(10) Pub. No.: US 2022/0183736 A1
(43) Pub. Date: Jun. 16, 2022

(54) POSITIONABLE, REMOVABLE SUCTION SYSTEM FOR USE DURING A SURGICAL PROCEDURE AND METHODS OF MAKING AND USING SAME

(71) Applicants: Mark McBride, Coronado, CA (US); Carola Romero, Coronado, CA (US)

(72) Inventors: Mark McBride, Coronado, CA (US); Carola Romero, Coronado, CA (US)

(21) Appl. No.: 17/119,728

(22) Filed: Dec. 11, 2020

(51) Int. Cl. A61B 18/00

(52) U.S. Cl. CPC A61B 18/00; A61B 2218/008

(57) ABSTRACT

The present invention is generally related to a suction system for use during surgical procedures. The suction system for use during surgical procedures utilizes a suction device that is postionable so that the suction device can be located in its most efficient position in order to capture as much smoke and odors as possible that are being produced at a surgical site. Furthermore, the suction system is also capable of being quickly and easily removed from the patient's surgical field so that the suction system can be quickly and easily located at another location on the patient's surgical field, if needed. Finally, the suction system includes widened head areas that create a wide suction area in order to further assist in capturing as much smoke and odors as possible that are being produced at a surgical site.

### FIELD OF THE INVENTION

The present invention is generally related to a suction system for use during surgical procedures. The suction system for use during surgical procedures of the present invention utilizes a suction device that is postionable so that the suction device can be located in its most efficient position in order to capture as much smoke and odors as possible that are being produced at a surgical site. Furthermore, the suction system of the present invention is also capable of being quickly and easily removed from the attachment at the margins of the surgical wound so that the suction system can be quickly and easily located at another location of the surgical field, if needed. Finally, the suction system includes widened head areas that create a wide suction area in order to further assist in capturing as much smoke and odors as possible that are being produced at a surgical site.

### BACKGROUND OF THE INVENTION

Prior to the present invention, as set forth in general terms above and more specifically below, it is known, to employ various types of suction systems for use during surgical procedures. Currently, a suction hose is conventionally connected to a conventional vacuum so that the suction hose is used to capture smoke and odors that are being produced at a surgical site. While these and other various suction systems for use during surgical procedures may have been generally satisfactory, there is nevertheless a need for a new and improved, positionable, removable suction system for use during surgical procedures.

It is a purpose of this invention to fulfill these and other needs in the art of suction system for use during surgical procedures in a manner more apparent to the skilled artisan once given the following disclosure.

The preferred positionable, removable suction system for use during surgical procedures, according to various embodiments of the present invention, offers the following advantages: ease of use; lightness in weight; durability; improved ability to remove the smoke and odors produced at a surgical site; the ability to position the suction system; the ability to easily remove the suction system; the ability to easily locate the suction system; increased suction area at the surgical site; the system is passive when positioned and does not require an assistant to continuously hold the device in place, and reduced noise produced by the suction system. In fact, in many of the preferred embodiments, these advantages are optimized to an extent that is considerably higher than heretofore achieved in prior, known suction systems for use during surgical procedures.

### BRIEF DESCRIPTION OF THE DRAWINGS

The above-mentioned features and steps of the invention and the manner of attaining them will become apparent, and the invention itself will be best understood by reference to the following description of the embodiments of the invention in conjunction with the accompanying drawings, wherein like characters represent like parts throughout the several views and in which:

FIG. 1 is a schematic, top view illustration of a positionable, removable suction system for use during surgical procedures, according to one embodiment of the present invention;

FIG. 2 is a schematic, bottom view illustration of the positionable, removable suction system for use during surgical procedures, according to one embodiment of the present invention; and

FIGS. 3a-3c are schematic illustrations of the suction head of the positionable, removable suction system for use during surgical procedures, according to one embodiment of the present invention.

### DETAILED DESCRIPTION OF THE PREFERRED EMBODIMENTS OF THE INVENTION

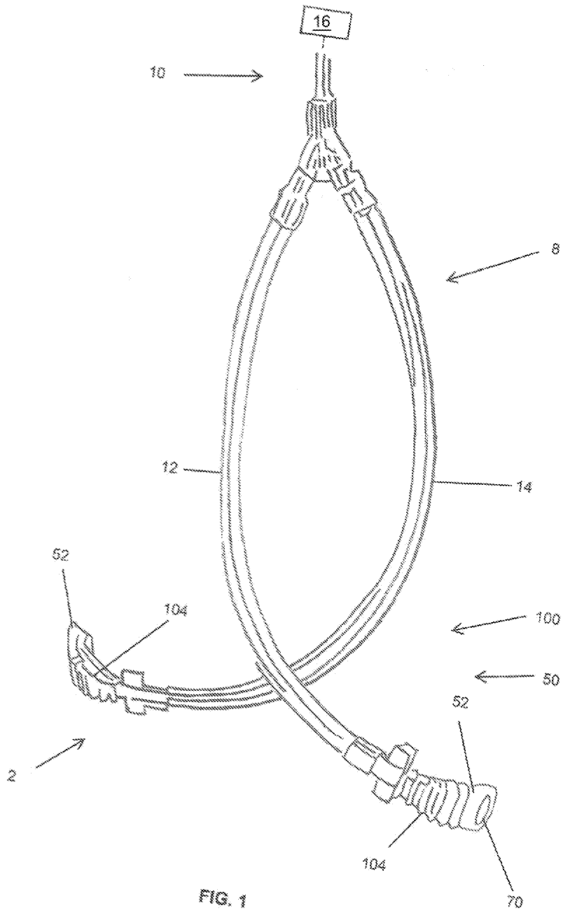

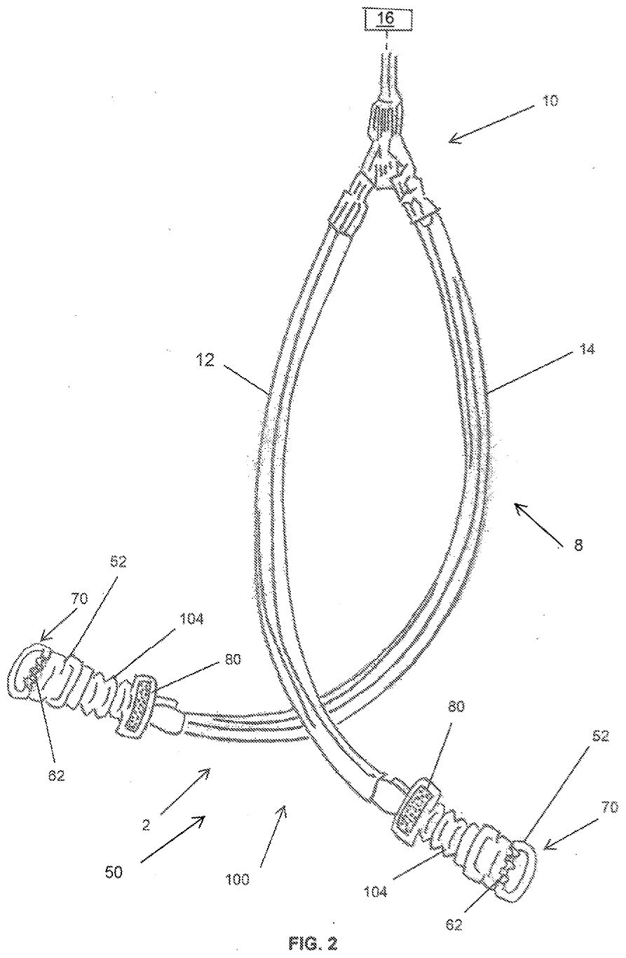

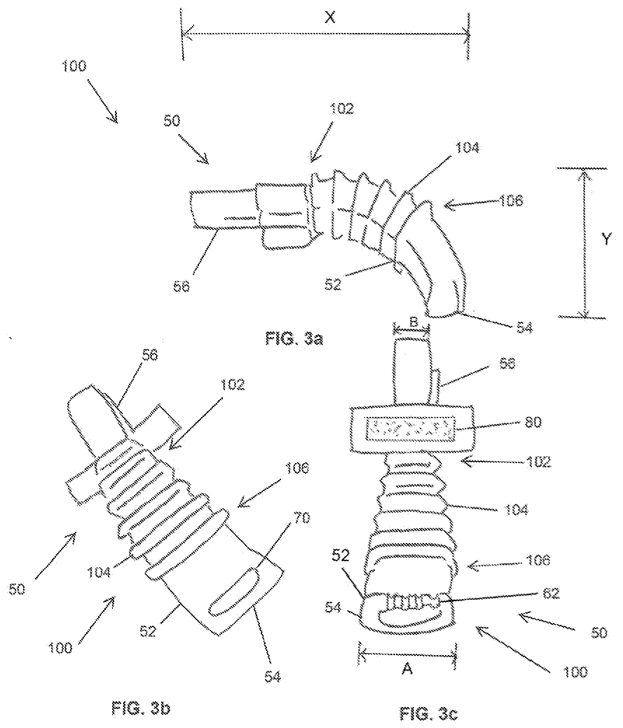

Referring now to FIGS. 1 and 2, there are illustrated a positionable, removable suction system 2 for use during surgical procedures. The positionable, removable suction system 2 for use during surgical procedures utilizes a suction device 50 that is postionable using a positioning device 100 (FIGS. 3a-3c) so that the suction system 2 can be located in its most efficient position in order to capture as much smoke and odors as possible that are being produced at a surgical site. Furthermore, the positionable, removable suction system 2 for use during surgical procedures is also capable of being quickly and easily removed from the surgical field so that the suction system 2 can be quickly and easily located at another location on the surgical field, if needed. Finally, the suction system 2 includes widened head areas in the suction device 50 that create a wide suction area in order to further assist in capturing as much smoke and odors as possible that are being produced at a surgical site.

With respect to positionable, removable suction system 2 for use during surgical procedures, suction system 2 includes a vacuum device 8 having vacuum source connector 10, suction hoses 12 and 14, and conventional vacuum source 16. Preferably, vacuum source connector 10 is constructed of any suitable, durable, high-strength, medical grade polymeric material. Preferably, suction hoses 12 and 14 are constructed of any suitable, durable, high-strength, flexible, medical grade polymeric material. Also, vacuum source connector 10 should be of such a size and length so that it is capable of adequately connecting to vacuum source 16 and suction hoses 12 and 14. Furthermore, suction hoses 12 and 14 should be of such a size and length so that they are capable of adequately providing the needed vacuum pressure to suction device 50 but also allow vacuum source 16 to be properly located away from the surgical procedure. It is to be understood that one end of each of the suction hoses 12 and 14 is conventionally connected to suction device 50, as will be described in greater detail later. Finally, it is to be understood that vacuum source 16 should be constructed so that it is able to provide the desired vacuum pressure to the suction device 50 so that suction device 50 is capable of capturing as much smoke and odors as possible that are being produced at a surgical site.

### Suction Device

As shown in FIGS. 3a-3c, positionable, removable suction system 2 for use during surgical procedures includes, in part, suction device 50. Suction device 50 includes, in part, suction head 52, suction head end 54, suction head connector 56, suction head ridges 62, suction head opening 70, and suction head fastener 80. It is to be understood that suction head 52, suction head end 54, suction head connector 56, and suction head ridges 62 are constructed of any suitable, durable, high strength, rigid, medical grade materials. It is to be understood that suction head connector 56 is used to conventionally connect suction head 52 to one end of each of the suction hoses 12, 14, as will be discussed in greater detail later. It is to be even further understood that suction head ridges 62 are conventionally formed on the end of each of the suction heads 52 at suction head end 54 by conventional techniques such as forming, molding, grinding or the like. Also, suction head 52, preferably, has a length of 1-2 cm along the direction of arrow (X) as shown in FIG. 3a and a curved distance (Y) of 1-2 cm as shown in FIG. 3a.

Another unique aspect of the present invention is that a width (A) of suction head 52 should be larger than a width (B) of suction head connector 56. In this manner, the wider size of section head 52 in comparison to section head connector 56 will further assist in capturing as much smoke and odors as possible that are being produced at a surgical site.

A unique aspect of the present invention is that that suction device 50 includes two (2) suction heads 52. Also, each suction device 50 is curved. In this manner, the use of the two (2) suction heads 52 and the curved nature of each of the suction device 50 allow suction devices 50 to be located on both sides of the surgical site in order to efficiently capture as much smoke and odors as possible that are being produced at a surgical site. Furthermore, the suction devices 50 create a passive suction system when positioned and the suction devices 50 do not require an assistant to continuously hold the suction devices 50 in place.

Another unique aspect of the present invention is the use of suction head ridges 62. As shown in FIGS. 1, 2, and 3c, the suction head ridges 62 are located on a portion of suction head end 54 in order to reduce the likelihood that suction head 52, in particular, suction head end 54 will create an undesirable seal on the area of the patient that is being operated upon if the suction head end 54 comes into close proximity with the area of the patient that is being operated upon.

With respect to FIGS. 1 and 3b, a further unique aspect of the present invention is illustrated. In particular, as shown in FIGS. 1 and 3b, suction head 52 also includes an opening 70. It is to be understood that opening 70 is formed in the top of suction head 52 by conventional techniques such as forming, cutting, drilling or the like. Opening 70 is used to reduce the vacuum noise that is created in suction head 52 by temporarily lowering the vacuum pressure at or near the suction head end 54. Furthermore, opening 70 makes it easier to vacuum more fragile things by reducing the vacuum pressure at or near the suction head end 54. Finally, opening 70 also acts as a safety valve, in that if a large item is accidentally vacuumed into suction head 52 and plugs suction head end 54, air will be drawn through opening 70 instead of burning out the motor in vacuum 16.

### Suction Head Fastening Device

As shown in FIGS. 1, 2 and 3c, positionable, removable suction system 2 also includes suction head fastener 80. Preferably, fastener 80 is constructed of any suitable hook and loop fastener, such as Velcro®. It is to be understood that fastener 80 is attached to suction device 50 by conventional fastening techniques such as adhesives, tape, or the like.

Another unique aspect of the present invention is the use of suction head fastener 80. In particular, suction head fastener 80 can be used to removably secure suction device 50 to a surgical drape (not shown) that is being used during a surgical procedure. In particular, fastener 80 is located over the connection between suction head connector 56 and end 106 of positioning device 100 in order to assist in retaining suction device 50 onto the surgical field.

A still further unique aspect of the present invention is that fastener 80 allows suction device 50 to be easily and quickly removed from a particular location on the surgical field and, if needed, easily and quickly retained on another location on the surgical field as long as the surgical field has other similar hook and loop fasteners located on the surgical field at the other surgical sites. For example, if the patient is going to have several minor procedures performed during one long surgical procedure and those minor surgical procedures are located at different places on the patient, the surgical field can be equipped with similar hook and loop (Velcro®) fasteners located at the different surgical sites so that the suction device 50 can be moved from one surgical site and located to and retained at the other surgical site in a quick and easy manner.

### Positioning Device

With respect to FIGS. 3a-3c, there is illustrated positioning device 100. Positioning device 100 includes, in part, vacuum tube end 102, adjustable (malleable) memory section 104, and suction head end 106. It is to be understood that vacuum tube end 102 and suction head end 106, preferably, are constructed of any suitable, durable, high strength, rigid, medical grade materials. Also, it is to be understood that adjustable (malleable) memory section 104, preferably, is constructed of any suitable, durable, high strength, flexible, medical grade materials. It is to be further understood that a positioning device 100 is formed as part of each of the suction heads 52, as will be described in greater detail later.

Another unique aspect of the present invention is that adjustable (malleable) memory section 104 is to be constructed so that positioning device 100 will be able to bend and remain in the bent position in order to allow the suction heads 52 to be located within the surgical site at the desired locations, as will be discussed in greater detail later.

### Setting-Up the Positionable, Removable, Suction System

In order to set-up the positionable, removable suction system 2 for use during surgical procedures, attention is directed to FIGS. 1-3c. The suction devices 50 are conventionally attached by suction head ends 56 to the suction hoses 12 and 14.

Once each of the suction hoses 12, 14 has been connected to a suction device 50, a suction head fastener 80 is then placed on suction device 50. As discussed earlier, the suction head fastener 80 assists in retaining suction device 50 on the surgical gown. Also, the suction head fastener 80 can be used to removably, secure each of the suction device 50 to the surgical field at locations proximate to the surgical site.

After the suction head fastener 80 has been placed on each of the suction devices 50, each of the suction hoses 12, 14 are conventionally attached to the vacuum source 16 through the use of vacuum source connector 10, as discussed earlier.

### Operation of Positionable, Removable, Suction System

Another unique aspect of the present invention is the use of positionable, removable suction system 2 for use during surgical procedures. As shown in FIGS. 1-3c, before the positionable, removable suction system 2 has been set-up, as discussed above, the surgical personnel determine where the surgical site (or sites) are to located on the patient. Once the surgical site (or sites) has been determined, the surgical personnel set-up the positionable, removable suction system 2. The surgical personnel then conventionally attach fasteners (not shown) to the surgical field/drape around the surgical site. As discussed above, these fasteners should be compatible with fastener 80 on suction device 50. For example, the fasteners located on the surgical gown may be conventional hook and loop type fasteners (Velcro®) and the fasteners 80 on suction device 50 can also be constructed of a similar hook and loop type fastener. In this manner, suction head fastener 80 can be quickly and easily secured to the surgical field at the desired surgical site and then quickly and easily removed and placed at another surgical site on the surgical field, if needed.

Once the fasteners have been attached to the surgical field, the suction head fastener 80 can then be removably attached to the fasteners on the surgical field, as discussed above. The suction devices 50 can then be located/positioned using positioning device 100 so that they are located in their most efficient position in order to capture as much smoke and odors as possible that are being produced at a surgical site.

Another unique aspect of the present invention is the use of positioning device 100. As discussed above, positioning device 100 provides for the precise placement of the suction head 52 with respect to the surgical site by allowing the surgical personnel to position each of the suction heads 52 so that each suction head 52 in its most efficient position in order to capture as much smoke and odors as possible that are being produced at a surgical site. The adjustable (malleable) memory section 104 provides the positioning device 100 with the ability to position the suction head 52 in a variety of positions and still be able to provide adequate vacuum pressure to the surgical site in order to vacuum away the smoke and odors being emitted at the surgical site during the surgical procedure.

As discussed above, a further unique aspect of the present invention is that a width (A) of suction head 52 should be larger than a width (B) of suction head connector 56. In this manner, the wider size of section head 52 in comparison to section head connector 56 will further assist in capturing as much smoke and odors as possible that are being produced at a surgical site.

As discussed earlier, once the surgical procedure has been completed at the surgical site and there is a need to perform another surgical procedure at another surgical site on the patient, surgical personnel turn off the vacuum source 16. The surgical personnel then simply remove the fastener 80 from the fasteners on the surgical field at the current surgical site. The surgical personnel then locate the fastener 80 over the fasteners on the surgical field at the next surgical site on the surgical field and conventionally attach the fasteners 80 to the next set of fasteners (not shown) on the surgical field at the next surgical site. The surgical personnel then adjust the position of the suction heads 52 at the new surgical site and turn the vacuum source 16 back on. The surgical personnel then can adjust, if needed, the position of the suction heads 52 using positioning device 100 with respect to the new surgical site so that suction heads 52 are in their most efficient positions in order to capture as much smoke and odors as possible that are being produced at a surgical site.

The preceding merely illustrates the principles of the invention. It will thus be appreciated that those skilled in the art will be able to devise various arrangements which, although not explicitly described or shown herein, embody the principles of the invention and are included within its spirit and scope. Furthermore, all examples and conditional language recited herein are principally intended expressly to be only for pedagogical purposes and to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents and equivalents developed in the future, i.e., any elements developed that perform the same function, regardless of structure.

This description of the exemplary embodiments is intended to be read in connection with the figures of the accompanying drawing, which are to be considered part of the entire written description. In the description, relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

All of the features disclosed in this specification may be combined in any combination. Thus, unless expressly stated otherwise, each feature disclosed is only an example of a generic series of equivalent or similar features.

It is to be understood that while the invention has been described in conjunction with the detailed description thereof, the foregoing description is intended to illustrate and not limit the scope of the invention, which is defined by the scope of the appended claims. Thus, from the foregoing, it will be appreciated that, although specific embodiments of the invention have been described herein for the purpose of illustration, various modifications may be made without deviating from the spirit and scope of the invention. Other aspects, advantages, and modifications are within the scope of the following claims and the present invention is not limited except as by the appended claims.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein or in the claims.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended and those skilled in the art will recognize, that the invention embraces and is also thereby described in terms of any individual member or subgroup of members of the Markush group.

Therefore, provided herein is a new and improved positionable, removable suction system for use during surgical procedures. The preferred positionable, removable suction system for use during surgical procedures, according to various embodiments of the present invention, offers the following advantages: ease of use; lightness in weight; durability; improved ability to remove the smoke and odors produced at a surgical site; the ability to position the suction system; the ability to easily remove the suction system; the ability to easily locate the suction system; increased suction area at the surgical site; the ability to create a passive system when the suction devices are positioned in that the system does not require an assistant to continuously hold the suction devices in place; and reduced noise produced by the suction system.

In fact, in many of the preferred embodiments, these advantages of ease of use, lightness in weight, durability, improved ability to remove the smoke and odors produced at a surgical site, the ability to position the suction system, the ability to easily remove the suction system, the ability to easily locate the suction system, increased suction area at the surgical site, the ability to create a passive system when the suction devices are positioned in that the system does not require an assistant to continuously hold the suction devices in place, and reduced noise produced by the suction system are optimized to an extent that is considerably higher than heretofore achieved in prior, known suction system for use during surgical procedures.

We claim:

1. A positionable, removable, suction system for use during a surgical procedure, wherein the system comprises:
a plurality of suction hoses, wherein each of the plurality of suction hoses has a first end and a second end;
a vacuum source connector operatively connected to each of the first ends of the plurality of suction hoses; and
a suction device operatively connected to each of the second ends of the plurality of suction hoses, wherein the suction device comprises;
a suction head,
a suction head connector located on one end of the suction head and operatively connected to each of the second ends of the plurality of suction hoses, and
a plurality of suction head ridges located on the other end of the suction head.

2. The positionable, removable, suction system, according to claim 1, wherein the system further comprises:
a vacuum source operatively connected to the vacuum source connector.

3. The positionable, removable, suction system, according to claim 1, wherein the suction device further comprises:
a suction head opening located adjacent to the suction head ridges.

4. The positionable, removable, suction system, according to claim 1, wherein the suction device further comprises:
a suction head fastener located adjacent to the suction head connector.

5. The positionable, removable, suction system, according to claim 1, wherein the suction device further comprises:
a positioning device located between the suction head connector and the plurality of suction head ridges, wherein the positioning device comprises;
a vacuum tube end located adjacent to the suction head connector,
an adjustable memory section located adjacent to the vacuum tube end, and
a suction head end located adjacent to the malleable memory section and the plurality of suction head ridges.

6. The positionable, removable, suction system, according to claim 4, wherein the suction head fastener further comprises:
a hook and loop fastener.

7. The positionable, removable, suction system, according to claim 1, wherein a width of the suction head is larger than a width of the suction head connector in order to assist in capturing as much smoke and odors as possible that are being produced at a surgical site.

8. A suction system, wherein the system comprises:
a plurality of suction hoses, wherein each of the plurality of suction hoses has a first end and a second end;
a vacuum source connector operatively connected to each of the first ends of the plurality of suction hoses; and
a suction device operatively connected to each of the second ends of the plurality of suction hoses, wherein the suction device comprises;
a suction head,
a suction head connector located on one end of the suction head and operatively connected to each of the second ends of the plurality of suction hoses, and
a plurality of suction head ridges located on the other end of the suction head.

9. The suction system, according to claim 8, wherein the system further comprises:
a vacuum source operatively connected to the vacuum source connector.

10. The suction system, according to claim 8, wherein the suction device further comprises:
a suction head opening located adjacent to the suction head ridges.

11. The suction system, according to claim 8, wherein the suction device further comprises:
a suction head fastener located adjacent to the suction head connector.

12. The suction system, according to claim 8, wherein the suction device further comprises:
a positioning device located between the suction head connector and the plurality of suction head ridges, wherein the positioning device comprises;
a vacuum tube end located adjacent to the suction head connector,
an adjustable memory section located adjacent to the vacuum tube end, and
a suction head end located adjacent to the malleable memory section and the plurality of suction head ridges.

13. The suction system, according to claim 11, wherein the suction head fastener further comprises:
a hook and loop fastener.

14. The suction system, according to claim 8, wherein a width of the suction head is larger than a width of the suction head connector in order to assist in capturing as much smoke and odors as possible that are being produced at a surgical site.

15. A method of constructing a positionable, removable, suction system for use during a surgical procedure, wherein the system comprises:
providing a plurality of suction hoses, wherein each of the plurality of suction hoses has a first end and a second end;
attaching a vacuum source connector to each of the first ends of the plurality of suction hoses; and
attaching a suction device to each of the second ends of the plurality of suction hoses, wherein the suction device comprises;
a suction head,
a suction head connector located on one end of the suction head and operatively connected to each of the second ends of the plurality of suction hoses, and
a plurality of suction head ridges located on the other end of the suction head.

16. The method, according to claim 15, wherein the system further comprises:
attaching a vacuum source to the vacuum source connector.

17. The method, according to claim 15, wherein the suction device further comprises:
a suction head opening located adjacent to the suction head ridges.

18. The method, according to claim 15, wherein the suction device further comprises:
attaching a suction head fastener to the suction head connector.

19. The method, according to claim 15, wherein the suction device further comprises:
a positioning device located between the suction head connector and the plurality of suction head ridges, wherein the positioning device comprises;
a vacuum tube end located adjacent to the suction head connector,
an adjustable memory section located adjacent to the vacuum tube end, and
a suction head end located adjacent to the malleable memory section and the plurality of suction head ridges.

20. The method, according to claim 15, wherein a width of the suction head is larger than a width of the suction head connector in order to assist in capturing as much smoke and odors as possible that are being produced at a surgical site.
